(19) United States
(12) Patent Application Publication
(10) Pub. No.: US 2024/0201216 A1
(43) Pub. Date: Jun. 20, 2024

(54) ASSEMBLY

(71) Applicant: Endress+Hauser Conducta GmbH+Co. KG, Gerlingen (DE)

(72) Inventors: Holger Lippert, Leisnig (DE); Thomas Pfauch, Leipzig (DE); Arne Kleinpeter, Neustadt an der Weinstrasse (DE); Maik Thormann, Leipzig (DE)

(21) Appl. No.: 18/528,988

(22) Filed: Dec. 5, 2023

(30) Foreign Application Priority Data
Dec. 16, 2022 (DE) 10 2022 133 611.5

(51) Int. Cl. G01N 35/10

(52) U.S. Cl. CPC G01N 35/1004; G01N 35/1097

(57) ABSTRACT

An assembly for receiving a sensor to measure at least one measurand of a medium in a container includes: a housing having an immersion portion including a first opening and a second opening opposite the first opening; a rotatable valve body which includes a first valve body opening and a second valve body opening opposite the first valve body opening and forms a channel, wherein a service chamber is formed between the first and second valve body openings, wherein, in a first position of the valve body, the first opening, the second opening, the first valve body opening and the second valve body opening collectively form an opening to enable a flow through the service chamber and, when the valve body is in a second position, to block the flow through the service chamber; and a head plate including a first flushing connection and a second flushing connection.

### CROSS-REFERENCE TO RELATED APPLICATION

The present application is related to and claims the priority benefit of German Patent Application No. 10 2022 133 611.5, filed Dec. 16, 2022, the entire contents of which are incorporated herein by reference.

### TECHNICAL FIELD

The invention relates to an assembly for receiving a sensor adapted to measure at least one measurand of a medium in a container.

### BACKGROUND

Assemblies, for example, retractable assemblies, are widely used in analytical measurement technology and process automation. They are used for removing sensors from the process, and thus from the medium, without interrupting the process, and for then reintroducing them into the process. The sensors are fastened in a dip tube and are moved axially by hand or automatically, for example, pneumatically, by means of a drive between a process position (e.g., measurement) and a service position (e.g., maintenance, calibration, flushing, probe exchange, etc.). These processes are executed after a certain period of time, depending on the drift of the measured value or the contamination of the measuring element. The sensors are used to measure one or more physical or chemical process variables.

A great variety of retractable assemblies are offered and marketed by the Endress+Hauser corporate group, for example, under the name “Cleanfit CPA871.” Information about them can be found on Applicant's website, for example, regarding the filing date under: http://www.endress.com/cpa871.

In such a retractable assembly, a sensor carrier including a sensor is moved in a linear fashion along the main axis of the assembly into a process, e.g., into the medium. The movement results in the transition from the service position (where sensor is isolated from the process, for example, for cleaning or calibration) into a measuring position (where sensor is in the process, sometimes also termed the process position). In the service position, the seal to the environment or to the process is effectuated, for example, by a pin with polymer seals. This region is traversed by the above-described linear movement from the service position into the measuring position or vice versa, resulting in high stress and signs of wear. Long travel paths require long sensor carriers. In this case, there is a risk of adhesion to the pipe, which can greatly stress the sealing system, especially in the case of polymers. The seals must thus be replaced frequently. Due to the process, the movement additionally stresses the sensor. Adhesions due to caking and/or crystallizing media, moreover, increase the risk of glass breakage during installation and removal of the sensor when using pH glass sensors.

The above-mentioned sealing system between the housing and the sensor or sensor holder (also called sensor carrier) provides a separation from the process chamber. The type of sealing system determines the way in which the sensor carrier travels between the two positions. The type of arrangement of a service chamber determines the type of flushing media supply. The “service chamber” is also referred to as a flushing chamber or calibration chamber.

Sealing rings are often used, which causes a purely linear movement of the sensor carrier. An example of this is the aforementioned CPA871 from Applicant. In this case, a flushing chamber is arranged offset in the axial direction, wherein, in one embodiment, the flushing chamber lies completely outside the process zone. In this case, the flushing medium can be supplied or discharged relatively easily via two connections.

When sealing disks are used, a rotational movement of the sensor carrier is still necessary in addition to the linear movement. Assemblies using this principle can also be purchased commercially. The flushing chamber is then accommodated inside the process zone. In order for the flushing medium to be supplied to and removed from the chamber inside the process zone, two separate channels must be present, each having two connections outside the process zone. Such an example is presented in DE 20 2007 006 784 U1.

These channels are often designed to be very thin due to the confined space available, given the installation situation (e.g., diameter up to 3 mm), which is disadvantageous, in particular when contaminants which are viscous or loaded with particles are to be removed (see also, above regarding adhesion).

A purely rotational movement (rotation/pivoting) is possible by means of the plug or ball valve principle. In such an assembly, it is possible to use the closed position as a service position and to provide it with an additional flushing connection and flush out everything which is in the through-opening of the valve body (in this case the plug). DE 10 2020 120 823 A1, which presents an assembly with a rotatable closing element, originates from Applicant.

### SUMMARY

The object of the invention is to overcome the disadvantages of the prior art. In particular, an assembly is to be provided which makes it possible to clean the sensor with flushing medium and flush it at the same time, continuing once the process has started, especially with sufficient flushing medium to prevent caking with undesired particles and media.

The object is achieved by an assembly for receiving a sensor which is designed to measure at least one measured variable of a medium in a process container, comprising a substantially hollow-cylindrical housing which is designed to connect the assembly to the process container via a process connection and comprises an immersion portion, at least portions of which are cylindrical, wherein the immersion portion comprises a first opening and a second opening that is opposite the first opening; a valve body, in particular a cylindrical and rotatable valve body, which is arranged in the immersion portion, at least in portions, extends along the longitudinal axis of the immersion portion and comprises, on the side remote from the process connection, a first valve body opening and a second valve body opening that is opposite the first valve body opening and forms a channel, wherein a service chamber is formed in a region between the first and second valve body openings, wherein, when the valve body is in a first position, the first opening, the second opening, the first valve body opening and the second valve body opening collectively form an opening, in particular the respective central points of the first opening, second opening, first valve body opening and second valve body opening are aligned and thereby enable a flow through the service chamber from the first opening, first valve body opening and second valve body opening to the second opening, and, when the valve body is in a second position, block a flow through the service chamber; the sensor comprising a sensitive element, wherein the sensor is arranged in the valve body such that the sensitive region is located between the valve body openings, and therefore in the service chamber, where the medium can flow against it; and a head plate that is arranged on the side of the process connection facing away from the medium, comprising a first flushing connection and a second flushing connection, wherein the first flushing connection opens into a first channel, wherein the first channel is formed between the valve body and the sensor, wherein the second flushing connection opens into a second channel, wherein the second channel is formed between the valve body and the immersion portion.

By using a movable valve body, assemblies can be made possible that have a flushing chamber (service chamber), which is arranged at the same height as the process medium inlet inside the process zone. This additionally avoids all other disadvantages of assemblies having linear guidance of the sensor carrier/valve body.

By using gaps between the valve body and the sensor carrier (or sensor, see below), which usually already exist by design, and between the valve body and the immersion housing as flushing channels, larger flow cross sections can be realized. As a result of the selected flow direction, larger flow cross sections are used for the contaminated/used flushing medium to prevent blockages due to particles or an excessive pressure drop due to a higher viscosity.

Due to the almost complete coverage of inner cavities with flushing medium, undetectable internal leaks are also eliminated.

An integrated check valve (see below) prevents process medium from passing into the flushing medium supply.

One embodiment provides that the assembly comprises a sensor carrier in which the sensor is arranged. The sensor carrier serves, for example, to protect the sensor or the outside world, for example if the sensor gets broken.

One embodiment provides that the sensor carrier comprises a thread, especially an internal thread, and the sensor, in particular having an external thread, is screwed thereinto.

One embodiment provides that the sensor carrier can be moved axially in the housing at least between a measuring/service position and an external position, i.e., can be removed from the assembly.

One embodiment provides that the sensor carrier can be moved manually, pneumatically or using a motor into and out of the measuring/service position.

One embodiment provides that the sensor carrier is connected to the housing by means of a quick-release connector, in particular a bayonet connector.

One embodiment provides that the sensor carrier is designed in accordance with the immersion depth in the service chamber.

One embodiment provides that the sensor carrier comprises a protective basket for the sensitive element of the sensor in the end region on the service chamber side.

One embodiment provides that the housing comprises a scraper seal, by means of which medium is scraped from the sensor during the movement of the sensor carrier out of the measuring/service position.

One embodiment provides that the first channel is formed between the valve body and the sensor carrier.

One embodiment provides that the assembly comprises a sealing insert which is arranged in the immersion portion between the sensor or sensor carrier and the inner housing wall of the immersion portion, comprises a first sealing insert opening and a second sealing insert opening so that, when the valve body is in the first position, a flow is made possible, and comprises at least one duct which connects the first channel to the second channel.

One embodiment provides that the head plate is designed in two parts.

One embodiment provides that the head plate is part of the housing.

One embodiment provides that the first channel and/or the second channel run along the circumference of the sensor or the sensor carrier over its entire length.

One embodiment provides that the valve body can be rotated by 90° or 45°.

One embodiment provides that the first channel comprises a check valve or a sealing sleeve which prevents medium from flowing back.

One embodiment provides that the housing comprises one or more seals which seal the service chamber from the environment.

One embodiment provides that a handle for moving the valve body is arranged thereon.

### BRIEF DESCRIPTION OF THE DRAWINGS

The present disclosure is explained in more detail with reference to the following figures, which include:

FIGS. 1a and 1b each show an assembly according to the present disclosure, each in a perspective view;

FIG. 2 shows the assembly in cross-section in the measuring position;

FIG. 3 shows the assembly in cross-section in the service position;

FIG. 4 shows a plan view of a head plate according to the present disclosure;

FIGS. 5a-5c show the assembly in cross-section in the service position together with the course of the flushing medium;

FIG. 6 shows another plan view of the head plate;

FIGS. 7a and 7b show detailed, cross-sectional perspective views of the assembly in the service or measuring position;

FIGS. 7c and 7d show horizontal cross-sectional plan views approximately at a height of the sensitive element of the sensor;

FIG. 7e shows a partial detailed view of a sealing insert according to the present disclosure; and

FIGS. 8a and 8b show a detailed cross-sectional view of arrangement of a check valve or a sealing sleeve according to the present disclosure.

### DETAILED DESCRIPTION

In the figures, the same features are labeled with the same reference signs. “Top,” “above,” and related terms within the meaning of this invention mean facing away from the measuring medium 14. “Bottom,” “below,” and related terms within the meaning of this invention mean facing the medium 14.

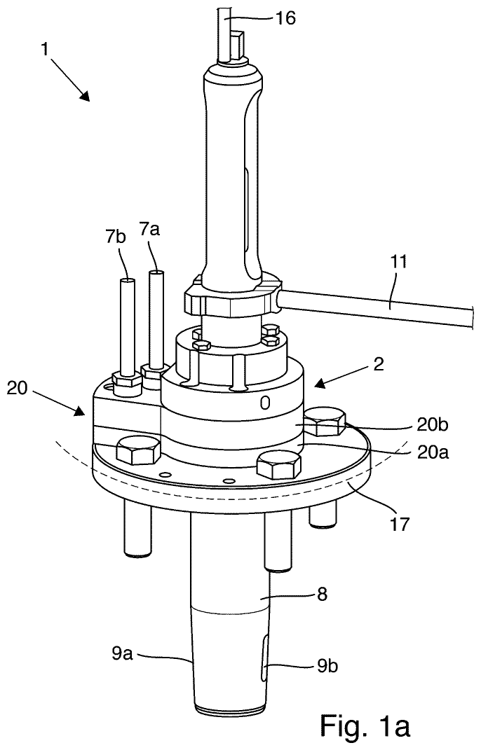

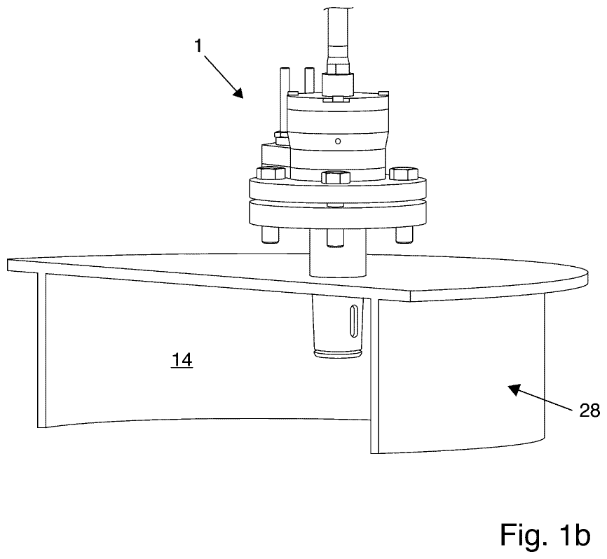

The assembly according to the present disclosure, as a whole, is given reference character 1 and is shown in FIG. 1a and FIG. 1b.

The assembly 1 comprises a substantially cylindrical housing 2, which is configured to connect the assembly 1 to a process container 28. The assembly 1 can be mounted directly in the process. The connection is done, for example, using standard screw, adhesive, welding or flange connections 17. The process container 28 is, for example, a pipeline or a basin.

The assembly 1 shown in FIG. 1a is connected to a container by means of the connection 17 in such a way that part of the housing 2, the immersion portion 8 (as described further below), projects into the process container 28, e.g., the edge of the container runs, for example, along the dashed line (FIG. 1a; see FIG. 1b for an installation condition). This is the case for attachment in a basin, for example. The immersion portion 8 is then permanently (e.g., continually) exposed to the medium. The medium 14 to be measured is located below the dashed line in the illustration illustrated in FIG. 1. The claimed assembly 1 is typically used for measuring liquids. For attachment to a pipe, the connection 17 is designed accordingly. The process container 28 can, for example, be a tank, boiler, tube, pipeline, or the like.

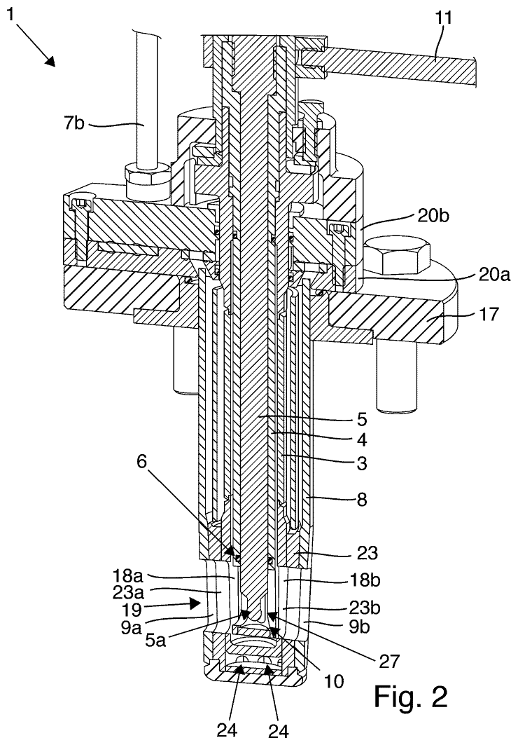

In an assembly 1 having a valve body 3 (explained in more detail below), said valve body 3 comprises two openings 9a, 9b which form a channel 27 (see FIG. 2). This is also referred to as a closing element. Characteristic of such an assembly is the way in which it is completely closed by rotating the valve body 3 by, for example, 90°. A rotational movement thus takes place. These assemblies can be equipped with actuators in order to automate opening and closing. By rotating the closing element 3 by 90°, medium 14 can thus flow through the assembly 1 or be prevented from doing so. The valve body 3 can thus be moved between two positions for “in process” or “in service”.

The valve body 3 is cylindrical. In one embodiment, the valve body 3 is conical or frustoconical, at least in portions, particularly around the lower end.

The immersion portion 8 of the housing 2 that is cylindrical, at least in portions, is permanently arranged in the medium 14 when the assembly 1 is fastened to the process container 28 as intended (when the container is designed as a basin; when designed as a pipe, the connection is made accordingly; see above). The immersion portion 8 comprises a first and second opening 9a, 9b through which medium 14 can flow in and out. The region of the assembly 1 between the openings 9a, 9b (or between the valve body openings 18a, 18b; see below) is referred to as the service chamber 10. By rotating the valve body 3, a flow through the service chamber 10 is released (first position of the closing element) or blocked (second position of the closing element). The space bearing reference sign “10” is in principle a sensor chamber—this becomes part of the process or media chamber when the assembly is in the measuring position and becomes the service chamber when the assembly is in the service position, namely through the connection for guiding the media into the various respective positions via the openings 18a and 18b of the valve body 3.

FIG. 1b shows a typical installation situation of the assembly 1.

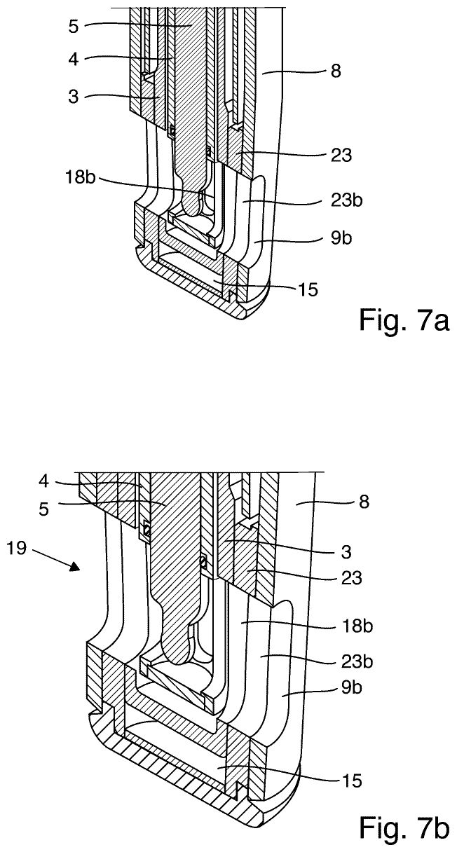

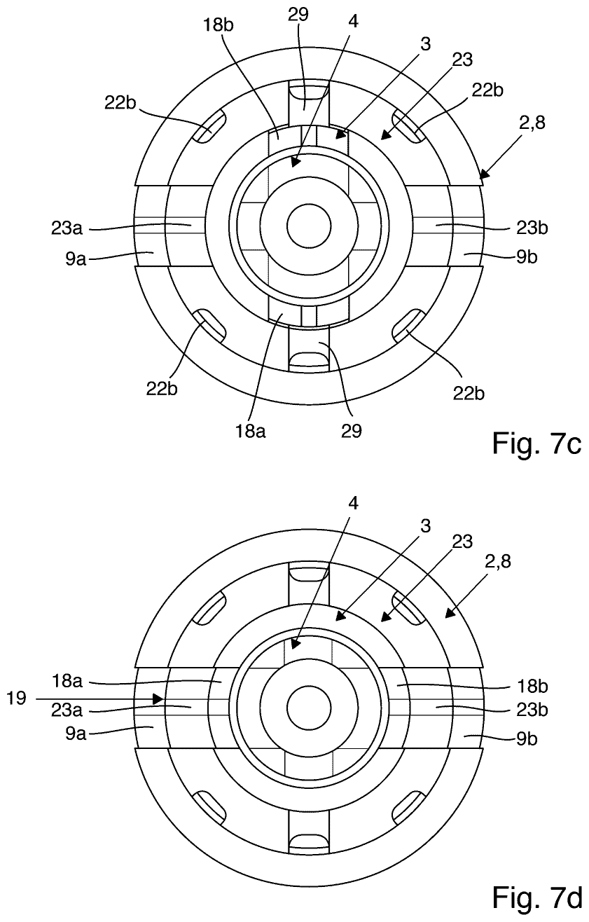

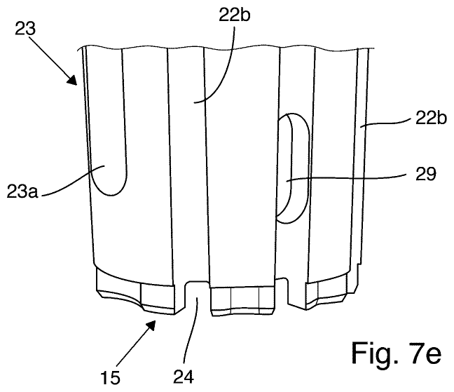

FIG. 2 and FIG. 7b show the assembly 1 in the open position (process position, measurement position, first position); FIGS. 2, 3, 4, 5-5c, 6 and 7a show the assembly 1 in the closed position (service position, calibration position, flushing position, second position).

The assembly 1 therefore comprises the movable valve body 3 in the housing 2, which extends along the longitudinal axis of the immersion portion 8, and, on the side remote from the process connection 17, comprises a first valve body opening 18a and a second valve body opening 18b, which is opposite the first valve body opening 18a. The service chamber 10 is formed in the region between the first and second valve body openings 18a, 18b. This is shown in FIG. 2, for example.

In the first position, the first opening 9a, the second opening 9b, the first valve body opening 18a and the second valve body opening 18b collectively form a, for example, cylindrical, opening 19; see for example FIG. 2 or FIG. 7b. The medium flows through the openings, particularly to and around the sensor 5, more precisely to the sensitive region 5a of the sensor 5 (see below regarding the sensor). In one embodiment, for example, the respective central points of the first opening 9a, second opening 9b, first valve body opening 18a, and second valve body opening 18b are aligned. The openings 9a, 18a or 9b, 18b are arranged on the “left” and “right”, respectively.

In the second position, a flow through the service chamber 10 is blocked by the valve body rotating by 90°, and the valve body opening 18a, b no longer “matching up” with the openings 9a, 9b. This is visible, for example, in FIG. 7a, wherein the valve body opening 18b is located “at the rear” in this view (i.e., the valve body opening 18a accordingly being located “at the front”), while furthermore the openings 9a, 9b are arranged on the “left” and “right”, respectively. FIG. 7c also shows this.

The assembly 1 comprises a sealing insert 23 which is arranged in the immersion portion 8 between the sensor 5 or sensor carrier 4 (see below) and the inner housing wall of the immersion portion 8. The sealing insert 23 comprises a first sealing insert opening 23a and a second sealing insert opening 23b so that a flow is made possible when the valve body 3 is in the first position. The openings 9a, 9b and the sealing insert openings 23a, 23b are not movable and are adjacent. The sealing insert 23 comprises at the bottom at least one duct 24, which connects a first channel 22a to a second channel 22b; see below.

The assembly 1 furthermore comprises a sensor carrier 4. The sensor 5 is arranged on or in the sensor carrier 4. The sensor carrier 4 is designed, for example, as a carriage; the sensor 5 then rests “against” the sensor carrier 4. The sensor carrier 4 may also be designed as a hollow-cylindrical component; the sensor 5 is then located “in” the sensor carrier 4. In both cases, an opening for the sensitive element 5a of the sensor 5 is at the bottom end.

The valve body 3, the sensor carrier 4 and the sealing insert 23 are designed as insert parts for the housing, more precisely for the immersion portion 8.

The sensor carrier 4 has, for example, an internal thread into which a sensor 5 having an external thread is screwed. In general, said sensor is connected at a first end (usually at the top) to the sensor carrier 4. A portion of the sensor carrier 4 is located in the housing 2, and a portion is located outside the housing 2.

In one embodiment, the sensor carrier 4 is movable in the housing between the measuring/service position and an external position. If the sensor carrier 4 is in the measuring/service position, the sensor 5 is arranged such that it is located with its sensitive element 5a in the service chamber 10.

The sensor 5 itself is accordingly not displaced but rather mounted in the assembly 1 by means of the sensor carrier 4. In addition to the simple (dis)assembly of the sensor 5, the object of the sensor carrier 4 is in particular to more easily service the process seal 6 to the sensor 5. As a result of the sensor carrier 4, it is furthermore possible to use sensors even at greater immersion depths. The sensor is, for example, 120 mm long. The short, less expensive sensors that are usually normally in stock can thus be installed, and a high immersion depth is nevertheless possible. In addition, the extension of the holder allows the immersion depth to be scaled. The sensor carrier 4, including the already preassembled sensor 5, can be mounted in the assembly 1 by means of a quick-release connector (e.g., bayonet). The carrier 4 can be inserted manually into the assembly 1. The movement may also take place by means of a pneumatic device or a motor.

The sensor carrier 4 is arranged in the housing 2 or in the valve body 3 such that the bottom end of the sensor 5 projects into the service chamber 10; the medium 14 accordingly flows around the sensor 5 in the first position of the valve body 3, and said sensor is separated from the medium 14 in the second position of the valve body 3. The sensor carrier 4 is adapted to the length of the sensor 5 in this case so that the sensor 5 always projects into the service chamber 10 by means of its sensitive region 5a. The sensor carrier 4 is designed in accordance with the immersion depth of the sensor 5 in the service chamber 10. At the bottom end region, the sensor carrier 4 comprises a protective basket for the sensitive element 5a of the sensor 5.

The sensor carrier 4 is connected to the housing 2, for example, by means of a quick-release connector, in particular a bayonet connector.

The sensor 5 within the meaning of this invention includes sensors for measuring one or more physical or chemical process variables. The sensor 5 comprises a sensitive region 5a, for example, for measuring the pH value, also via an ISFET, redox potential, absorption of electromagnetic waves in the medium 14, for example, with wavelengths in the UV, IR, and/or visible ranges, oxygen, conductivity, turbidity, concentration of metal and/or non-metal materials, or temperature. The sensor 5 is connected to a cable 16. The cable 16, in turn, is connected to a transmitter (not shown). The sensor 5 is designed as, for example, as a digital sensor with a microcontroller and memory; especially, the sensor 5 is an inductive sensor; especially, the sensor 5 supports plug & play with the transmitter connected thereto. Applicant sells such sensors under the name “Memosens.” The sensor 5 has a diameter of 12 mm, for example.

The sensor carrier 4 can be made from different materials, for example, steel or stainless steel. Also possible, for example, in the chemical industry, are very resistive materials. The sensor carrier 4 may accordingly also be made of a plastic, such as polyether ether ketone (PEEK), polytetrafluoroethylene (PTFE), a perfluoroalkoxy polymer (PFA), another plastic or resistive metals, such as Hastelloy. A ceramic may also be used. Especially, the closing element is made of a ceramic. Another option is the use of one or more coatings of the aforementioned polymers. The same applies to the housing 2, the immersion portion 8, or the valve body 3.

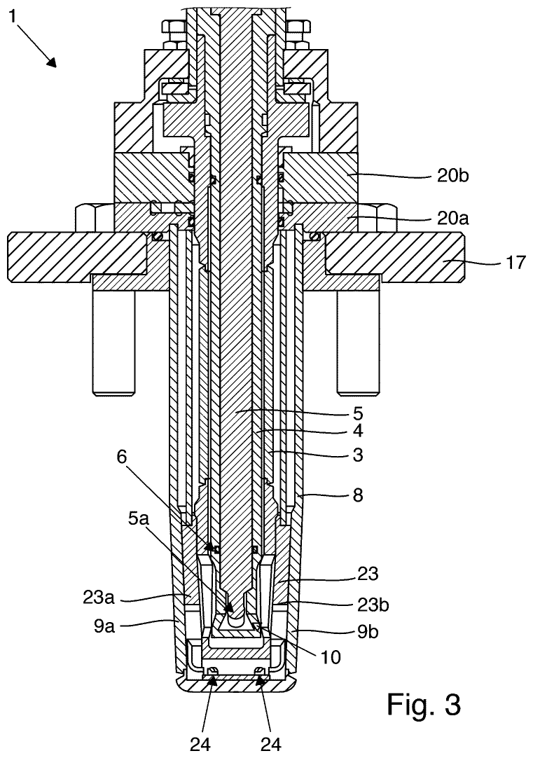

One or more seals 6 are arranged on the sensor carrier 4 and seal the flow region from the environment; see, for example, FIG. 2 or FIG. 3. “Environment” is understood to mean everything outside the process container and outside the assembly.

A handle 11 is attached to the valve body 3. As a result, the valve body 3 can be rotated easily. The valve body 3 may also be operated pneumatically or electrically, for example, with a swivel drive.

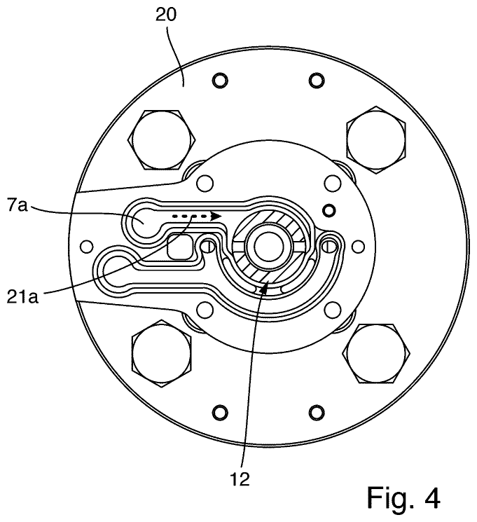

The second position or the flushing with flushing or calibration medium is demonstrated below with reference to FIGS. 3-6. FIG. 3 shows the assembly 1 in cross-section in the service position. FIGS. 4-6 show the course of the flushing medium, which is identified by arrows.

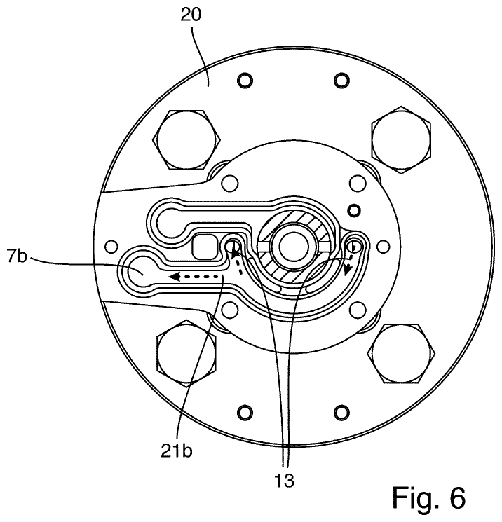

The flushing media enters the assembly 1 via the corresponding inlet 7a above the first flushing connection 21a of the head plate 20; see FIG. 4. The first flushing connection 21a and the second flushing connection 21b (see below, FIG. 6) are designed in the manner of a channel or tube and/or can be part of a corresponding molded seal in or on the head plate.

The head plate 20 is designed in one or two parts, in the present embodiment, in two parts with a first and a second part 20a, 20b. The head plate 20 can also be part of the housing 2.

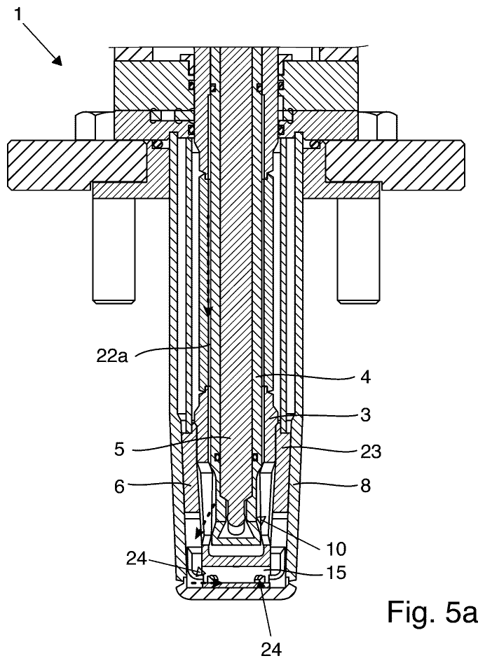

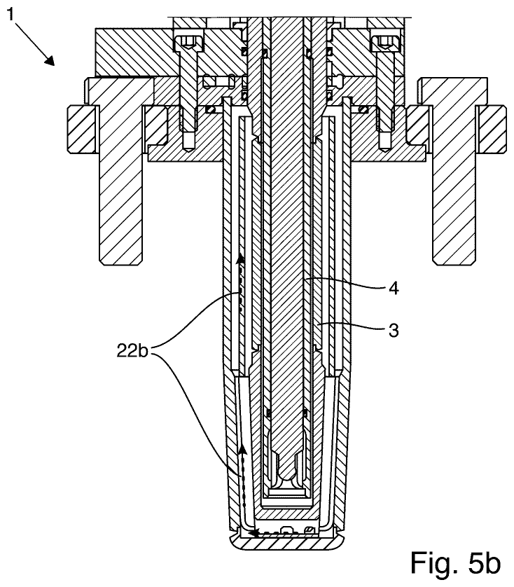

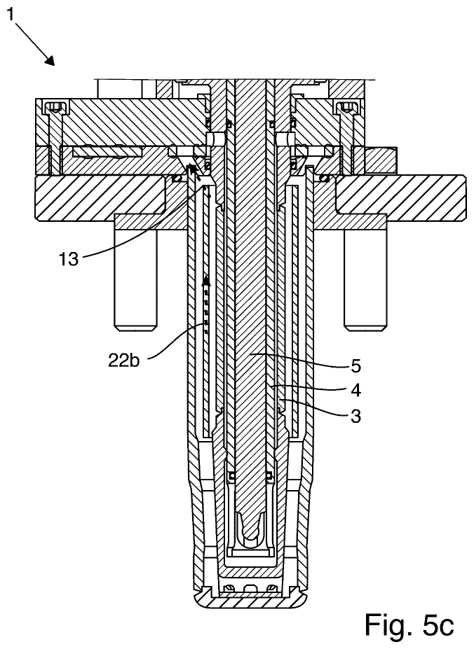

Via the inlet 7a and the flushing connection 21a, the flushing medium passes through transverse bores 12 and into a first channel 22a. The first channel 22a is formed between the valve body 3 and sensor 5; see FIG. 5a. In one embodiment, this is formed between the valve body 3 and sensor carrier 4 (which is arranged between the valve body 3 and sensor 5; see above). The main body 20 has corresponding bores 12 in the form of an inlet which open into the first channel 22a.

The first channel 22a and/or the second channel 22b are formed along the circumference of the sensor 5 or the sensor carrier 4 (depending on the embodiment), specifically in one embodiment, over the entire length thereof.

The flushing medium is accordingly guided downward between the sensor carrier 4 (or sensor 5) and valve body 3. The medium then enters the service chamber 10 at the measuring sensor 5 contaminated with process medium and is further discharged through the sealing insert 23 via one or more deflections 24 when the valve body is in the second position, as shown (service position; flushing position; e.g., FIGS. 5a, 7a, 7c). The medium then travels downward, guided by the sealing insert 23, into a chamber 15 between the valve body 3 and immersion portion 8. There, the flushing medium is deflected via one or more line channels 24 and guided upwards via the sealing insert 23 or the immersion portion 8 in the form of the second channel 22b in the further course between the immersion portion 8 and the valve body 3; see FIG. 5b or FIG. 7c. For example, four second channels 22b are present. These can be part of the sealing insert, as shown in FIG. 7c, 7d, or part of the immersion insert 8.

FIG. 7e shows a cutout of the sealing insert.

Flushing medium flows downwards through the channels 22a between the sensor 5 (or sensor carrier 4) and valve body 3, through the service chamber 10, via the valve body openings 18a, 18b and deflections 29 into a small chamber 15, and passes upwards via the line channels 24 into the second channels 22b.

Finally, the flushing medium is guided via bores 13 in the form of an outlet into the base plate 20; see FIG. 5c. The flushing medium is collected in the second flushing connection 21b and guided to the flushing medium outlet 7b of the head plate 20; see FIG. 6.

FIGS. 7a and 7b show a cutout of the assembly 1 in cross section in the service or measuring position. Likewise, FIGS. 7c and 7d show the assembly in the service or measuring position. In direct comparison, the position of the valve body 3 can be seen, for example, with reference to the valve body opening 18b, which is at the rear in FIG. 7a and on the right in FIG. 7b. A rotational movement of 90° (either to the right or left) was accordingly performed. In FIG. 7c, the valve body opening 18a, 18b is at the top or bottom (service position). In FIG. 7d, the valve body opening 18a, 18b is released in the “left-to-right direction”, the channel to the sensor (not shown) is free, and the total opening 19 is present (measurement position).

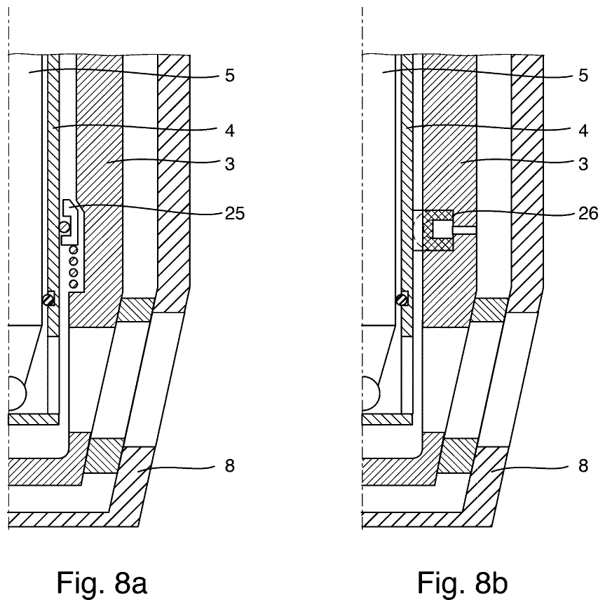

If the flushing media supply is activated, when the valve body 3 is in the process position, the flushing medium can be diverted into the process. The multiport valve function is accordingly provided. Otherwise, the possibility exists of process medium pushing into the flushing medium side via the flushing media supply. This can be solved by an external check valve in the flushing media supply or an integrated check valve 25, which sits in the shaft between the valve body 3 and sensor carrier 4 or sensor 5 or which is accommodated in the flushing flange. This is shown in FIG. 8a.

As a further embodiment, the following is conceivable: an additional valve body, which is spring-actuated, is always arranged so as to be sealed with respect to the sensor carrier 4 or sensor 5 and has a resilient or fixed mating seat of the valve on the valve body 3. Another embodiment of the check valve is the configuration that a resilient sealing sleeve 26 is seated in the valve body 3 or on the sensor carrier 4 and pressure medium (air or liquid) is applied thereto through corresponding holes. This is shown in FIG. 8b. The gap between the sensor 5 and the sensor carrier 4 is also possible as a channel if the sensor carrier is designed as a separate component, and thereby expands and closes a sealing gap between the sensor/sensor carrier 5/4 and valve body 3. This is also due to the fact that the sleeve 26 is seated in the valve body 3, is connected to the flushing medium via a transverse bore, and is subjected to a pressure in the form of a sealing pressure applied via the flushing media discharge.

An assembly is accordingly shown in which the flushing fluid is routed from the inside to the outside (the opposite fluid routing is also possible without an inventive step). The flushing fluid routing is selected such that all internal cavities can be flushed, and the flushing media are supplied via a special plate-like flushing flange to reduce installation space, and the possibility exists of flushing into the process chamber. The assembly can contain a check valve, which is arranged in such a way that misrouting of process medium is prevented.

Claimed is:

1. An assembly for receiving a sensor adapted to measure at least one measurand of a medium in a process container, the assembly comprising:
a substantially hollow, cylindrical housing configured to connect the assembly to the process container via a process connection and including an immersion portion, which is cylindrical at least in portions, wherein the immersion portion comprises a first opening and a second opening opposite the first opening;
a rotationally movable valve body, at least portions of which are disposed in the immersion portion, which valve body extends along a longitudinal axis of the immersion portion and, on a side remote from the process connection, comprises a first valve body opening and a second valve body opening opposite the first valve body opening, thereby forming a channel,
wherein a service chamber is defined in a region between the first and second valve body openings,
wherein, in a first position of the valve body, the first opening, the second opening, the first valve body opening, and the second valve body opening collectively define an opening,
in particular, the corresponding central points of the first opening, the second opening, the first valve body opening, and the second valve body opening are aligned as to enable a flow through the service chamber from the first opening, first valve body opening, and second valve body opening to the second opening and, when the valve body is in a second position, to block the flow through the service chamber;
the sensor comprising a sensitive element, wherein the sensor is disposed in the valve body such that the sensitive element is disposed between the first and second valve body openings and, accordingly in the service chamber, such that the medium can flow against the sensitive element; and
a head plate arranged on the side of the process connection facing away from the medium, the head plate comprising a first flushing connection and a second flushing connection,
wherein the first flushing connection opens into a first channel, wherein the first channel is defined between the valve body and the sensor,
wherein the second flushing connection opens into a second channel, wherein the second channel is defined between the valve body and the immersion portion.

2. The assembly according to claim 1, comprising a sensor carrier in which the sensor is disposed.

3. The assembly according to claim 2, wherein the first channel is defined between the valve body and the sensor carrier.

4. The assembly according to claim 2, comprising a sealing insert disposed in the immersion portion between the sensor or sensor carrier and an inner housing wall of the immersion portion, the sealing insert comprising:
a first sealing insert opening and a second sealing insert opening configured as to enable a flow in the first position of the valve body; and
an at least one line channel, which connects the first channel to the second channel.

5. The assembly according to claim 1, wherein the head plate is designed in two parts.

6. The assembly according to claim 1, wherein the head plate is part of the housing.

7. The assembly according to claim 1, wherein the first channel and/or the second channel extend along a circumference of the sensor or the sensor carrier over its entire length.

8. The assembly according to claim 1, wherein the valve body is rotatable by 90° or 45°.

9. The assembly according to claim 1, wherein the first channel comprises a check valve or a sealing sleeve configured to prevent the medium from flowing back.

10. The assembly according to claim 1, wherein the housing comprises one or more seals configured to seal the service chamber from the environment.

11. The assembly according to claim 1, wherein a handle is arranged on the valve body as to enable rotating the valve body.
